(19) United States
(12) Patent Application Publication
(10) Pub. No.: US 2008/0081788 A1
(43) Pub. Date: Apr. 3, 2008

(54) Diagnosis and treatment for immunoglobulin E (IgE) implicated disorders

(76) Inventor: Binie V. Lipps, Bellaire, TX (US)

Correspondence Address: John R. Casperson, P.O. Box 36369, Pensacola, FL, 32516-6369, US

(21) Appl. No.: 11/904,899

(22) Filed: Sep. 28, 2007

Related U.S. Application Data
(63) Continuation-in-part of application No. 10/047,945, filed on Jan. 14, 2002, now abandoned.
(63) Continuation-in-part of application No. 11/710,738, filed on Feb. 24, 2007.
Application No. 11/710,738 is a division of application No. 10/047,945, filed on Jan. 14, 2002, now abandoned.

(51) Int. Cl. A61K 38/00; A61P 11/06; A61P 37/02; A61P 7/12

(52) U.S. Cl. 514/14; 514/15; 514/16; 514/17

(57) ABSTRACT

Human saliva is used as a non-invasive source instead of invasive blood serum plasma for detection and assay of endogenously present proteins; nerve growth factor (NGF), myoglobin, Insulin, adenosine deaminase (ADA), including immunoglobulin E (IgE). It was discovered that people having high levels of IgE, show high levels in comparison to the normal controls of NGF, myoglobin, insulin and ADA, disrupting the homeostasis for these proteins. Oral administration of a synthetic peptide LT-10 disclosed in U.S. Pat. No. 5,576,297 having sequence L K A M D P T P P L reduces free IgE level in humans and brings other proteins int homeostasis, for example, NGF, myoglobin, insulin and ADA and possibly other proteins and cytokines. Composition of synthetic LT-10 is advocated as a treatment for IgE implicated disorders such as asthma, depression and various types of autoimmune diseases, such as erythematosus (SLE); Rheumatoid arthritis Sjogren's syndrome; Reiter's syndrome; Diabetes mellitus (insulin-dependent); Graves' disease; Addison's disease; Hodgkin's disease, etc.

### CROSS REFERENCE TO RELATED APPLICATIONS

This application is a continuation in part of application Ser. No. 10/047,945 fed Jan. 14, 2002, now ______ and/or a continuation in part of application Ser. No. 11/710,738 filed Feb. 24, 2007, now ______ which was a division of application Ser. No. 10/047,945 filed Jan. 14, 2002, now ______.

### SPECIFICATION

1. Field of the Invention

In one aspect, this invention relates to the introduction of use of saliva as a non-invasive source for detection and assay of endogenously present proteins, for example, nerve growth FACTOR (NGF) myoglobin, Insulin, adenosine deaminase (ADA), a most importantly immunoglobulin E (IgE). In another aspect, the invention relates to the treatment of human disorders characterized by elevated fee IgE levels by the administration of a peptide to reduce the level.

2. Discussion of Prior Art

U.S. Pat. No. 5,714,341, issued Feb. 3, 1998 discloses at col. 5; lines 15-34 “The method of the invention is useful for facilitating diagnosis of infection and other disease states in human subjects by detecting the presence of a disease-related analyte that is not ordinarily present or that is elevated above its normal level. The method also contemplates detection of disease states associated with reduced levels of substances that are secreted in the saliva of healthy subjects. She method may also find use for detecting drug abuse, or for verifying patient compliance in drug therapy. The method is compatible with a variety of oral fluid analytes. Such analytes include, but are not limited to, drugs or drug metabolites such as cocaine, benzoylgonine, cotinine, amphetamine, or methamphetamine, viral and bacterial antigens, such as antigens derived from hepatitis B, HIV-1 and HIV-2, immunoglobulins, such as particular immunoglobulin classes (e.g. IgG and IgM) or immunoglobulins specific against viral or bacterial pathogens, including immunoglobulins specific for hepatitis A, measles, rubella, mumps, and Heliobacter pylori; and hormones or hormone metabolites, such as B-hCG.”

U.S. Pat. No. 5,792,605 issued Aug. 11, 1998 discloses at column 1, lines 4-9 “This invention relates to methods of assay of immunoglobulin in saliva bodily fluid and particularly to a method of determining quantitatively the presence or amount of Hepatitis A virus specific IgG antibodies in saliva specimens.”

U.S. Pat. No. 5,965,709 issued Oct. 12, 1999. discloses at column 5, lines 15-17: “IgE antagonists include IgE variants, IgE peptide antagonists, peptidomimetics and other small molecules.” and at column 32, lines 51-58: “The IgE antagonists of the invention are used in therapies for the treatment or prophylaxis of allergies, although the IgE antagonist subgroup which bears cytotoxic functionalities is not considered suitable for therapy since it could lead to degranulation of mast cells and basophils. Otherwise, the IgE antagonists typically are administered to a patient who is known to be sensitized to an allergen, preferably prior to an acute allergic response.”

U.S. Pat. No. 6,172,213 issued Jan. 9, 2001 discloses at column 14, lines 47-57: “As used herein, the term “IgE-mediated disorder” means a condition or disease which is characterized by the over-production and/or hypersensitivity to the immunoglobulin IgE. Specifically it should be construed to include conditions associated with anaphylactic hypersensitivity and atopic allergies, including for example: asthma, allergic rhinitis & conjunctivitis (hay fever), eczema, urticaria and food allergies. However, the serious physiological condition of anaphylactic shock, usually caused by bee or snake stings or parental medication is also encompassed under the scope of this term.”

U.S. Pat. No. 6,271,390 issued Aug. 7, 2001 discloses at column 2, lines 28-62: “Consequently, new therapeutic approaches which target the underlying cause rather than the cascade of symptoms would be highly desirable. Asthma and allergy share a common dependence on IgE-mediated events. Indeed, it is known that excess IgE production is the underlying cause of allergies in general and allergic asthma in particular (Duplantier and Cheng, Ann. Rep. Med. Chem. 29:73-81 (1994)). Thus, compounds that lower IgE levels may be effective in treating the underlying cause of asthma and allergy. [N]one of the current therapies eliminate the excess circulating IgE. The hypothesis that lowering plasma IgE may reduce the allergic response, was confirmed by recent clinical results with chimeric anti-IgE antibody, CGP-51901, and recombinant humanized monoclonal antibody, rhuMAB-E25. Indeed, three companies, Tanox Biosystems, Inc., Genentech Inc. and Novartis AG are collaborating in the development of a humanized anti-IgE antibody (BioWorld.RTM. Today, Feb. 26, 1997, p. 2) which will treat allergy and asthma by neutralizing excess IgE. Tanox has already successfully tested the anti-IgE antibody, CGP-51901, which reduced the severity and duration of nasal symptoms of allergic rhinitis in a 155-patient Phase II trial (Scrip #2080, Nov. 24, 1995, p. 26). Genentech recently disclosed positive results from a 536 patient phase-II/II trials of its recombinant humanized monoclonal antibody, rhuMAB-E25 (BioWorld.RTM. Today, Nov. 10, 1998, p. 1). The antibody, rhuMAB-E25, administered by injection (highest dose 300 mg every 2 to 4 weeks as needed) provided a 50% reduction in the number of days a patient required additional “rescue” medicines (antihistamines and decongestants), compared to placebo. An NDA fling for this product is projected to be in the year 2000. The positive results from anti-IgE antibody trials suggest that therapeutic strategies aimed at IgE down-regulation may be effective.” and at column 37, line 55 through column 38, line 4: “Suppression of IgE Response [T]he inhibitory activity of the small molecules of the present invention were assayed using both the ex vivo and in vivo assays as described above. All of the compounds presented above were active in suppressing the IgE response. In the ex vivo assay, compounds in genuses I-XI produced 50% inhibition at concentrations ranging from 1 pM to 10 .mu.M. In the in vivo assay, the compounds were effective at concentrations ranging from less than about 0.01 mg/kg/day to about 25 mg/kg/day, when administered in divided doses (e.g., two to four times daily) for at least two to seven consecutive days. Thus, the small molecule inhibitors of the present invention are disclosed as being useful in lowering the antigen-induced increase in IgE concentration, and consequently, in the treatment of IgE-dependent processes such as allergies in general and allergic asthma in particular.”

US 2002/0004019 published Jan. 10, 2002 titled “Saliva Testing and Confirmation Device” discloses at paragraphs 45: “In addition, testing of salivary specimens has not yet been extensively developed. Blood and urine samples have for long been the primary fluids used for testing for disease as well as for evidence of substance abuse. However, it is now known that human saliva carries lymphocytes, plasma cells and immunoglobulins that are directly related to the immunoglobulins found in the blood. In addition, saliva carries immunoglobins that are believed to be peculia to saliva, for example, the antibody known as secretory IgA. [B]ecause of the association between immunoglobulins of the blood and saliva, as well as the occurrence of secretory IgA, antigen-antibody tests have been conducted on salivary fluid to assess the value of such tests as screening tools for disease.”U.S. Pat. No. 6,699,472, issued Mar. 2, 2004 discloses at col. 1, lines 18-26 “This invention relates to amino acid sequence variant anti-IgE antibodies and to polypeptides containing IgE sequences, especially IgE antagonists and to polypeptides capable of differential binding to FcεERI and FCεRII. [I]gE is a member of the immunoglobulin family that mediates allergic responses, such as asthma, food allergies, type 1 hypersensitivity and the familiar sinus inflammation suffered on a widespread basis.”

U.S. Pat. No. 7,101,851 issued Sep. 5, 2006 discloses at column 3, lines 31-50: “The binding of antibodies the Fc portion of IgE can inhibit binding of IgE to receptor (Pasta et al., (1993) J. Immunol. 151:2623 2632; Kolbinger et al., (1993) Protein Engineering 6:971 980) and can reduce free IgE levels in vivo (Saini et al., (1999)J. Immunol. 162:5624 5630). It has also been reported that certain peptides, designed to mimic a portion of the Fc.epsilon.RI receptor, can bind to the Fc portion of IgE and inhibit IgE binding to receptor (McDonnell et al., Nature Struct. Biol. 3:419425; McDonnell et al., (1997) Biochem Soc. Trans. 25:387 392). [P]resta et al. (1-993) J. Immunol. 151:2623 2632 disclose a humanized anti-IgE antibody that prevents the binding of free IgE to Fc.epsilon.RI but does not bind to Fc.epsilon.RI-bound IgE. Clinical studies of allergic individuals using an anti-IgE monoclonal antibody have been reported (Jardieu and Fick (1999) Intl. Arch. Allergy Immunol. 118:112 115). [A]s a result, molecules which block the binding of IgE to Fc.epsilon.RI, the IgE high affinity receptor would also be expected to have efficacy in the treatment of IgE-mediated disorders.” and at column 14, lines 40-51: An “effective amount” is at least the minimum concentration of IgE receptor antagonist peptide which prevents, lessens or causes to lessen the ability of said peptide to bind to the high affinity gE receptor (Fc.epsilon.RI). A “therapeutically effective amount” is at least the minimum concentration of IgE receptor antagonist peptide which attenuates or eliminates a pathological symptom or improves a pathological condition associated with an IgE-mediated disorder. For example, quantitative levels of inflammatory mediator(s) (e.g., histamine), fee IgE, IgE bound to the high affinity receptor, inflammation, tissue injury or the amount of leukocyte trafficking.”

### BACKGROUND OF THE INVENTION

For humans, immunoglobulins an other proteins are almost always assayed from serum. The use of saliva for assaying endogenous proteins would have several advantages over the current practice and use of serum. Saliva collection is non invasive, while blood collection for serum is invasive. Saliva collected in a tube can be centrifuged immediately to get rid of cells, while blood requires clotting time before it can be centrifuged to separate serum. Saliva proteins can be assayed by our discovered and perfected simple antigen antibody reaction test, by our discovered and perfected simple antigen antibody Enzyme-linked Immunosorbent (ELISA) test, whereas an assay of proteins from serum requires sandwich type ELISA, which is more complicated. It requires more time and reagents. In case of saliva the controls for ELISA have negligible background, whereas for serum the background noise has to be monitored carefully. Therefore, considering the above points, the use of saliva as a source to assay proteins, which can be done by a simple ELISA test with reproducible results, would be very desirable as high levels of proteins in saliva correlates with high levels in the serum

We have found that an elevated level of free IgE is implicated in (1) Type II diabetes (2) Depression (3) various types of Autoimmune diseases and (4) Asthma. It was revealed that the level of IgE in patients of these disorders is several times higher than the control normal individuals. We have also found that high levels of free IgE causes disruption in the homeostasis of endogenously present other proteins such as nerve growth factor, myoglobin, insulin and Adenosine deaminase. We believe that such disruption in homeostasis for NGF, myoglobin, insulin and ADA may be manifesting the symptoms for these disorders. For example, a high level of myoglobin may be implicated with a heart problem; a high level of insulin may indicate involvement of pancrease. It is known that a high level of ADA is due to asthma and involvement of lungs and diabetes.

A reagent to reduce elevated free IgE level in humans would be desirable. It has been proposed to use monoclonal antibodies against IgE (mono-anti-IgE) to reduce IgE level in asthma patients. However, a large protein molecule of mono-anti-IgE would be effective only by injection, and is costly. Further, administration of monoclonal antibody is a passive process of immunization. The life period of such passive antibody is a limited short period. Also, excess monoclonal antibody, not bound to free IgE, is liable to generate anti-anti-IgE or anti-idiotypic antibody which can interfere with treatment.

A small therapeutic molecule having low molecular weight which can be given orally would be very desirable.

### SUMMARY OF THE INVENTION

We have discovered that IgE and certain other endogenous protein serum levels in humans can be determined from saliva, and that IgE present is serum is also present at lesser concentrations in saliva.

We have found that free IgE serum levels in humans can be reduced by treatment with a low-molecular weight peptide.

We have found that a reduction in free IgE serum levels in humans brings concomitant reduction in certain other serum proteins which are disease and/or risk indicators.

We have found that the low molecular weight peptide is effective for the purpose of lowering free IgE in serum when given orally.

The low molecular weight employed in the invention is the synthetic LTNF described in U.S. Pat. No. 5,576,297 (19%) “Embodiments of Natural and Synthetic Lethal Toxin Neutralizing Factors (LTNFs) and their utility as treatment for Envenomation” and U.S. Pat. No. 5,744,449 (1998) “Lethal Toxin Neutralizing Factors.” The disclosures of these patents are incorporated by reference herein. After identify the active domain of natural LTNF, synthetic LTNF designated as LT-10 was made using ten amino acids having a sequence from the N-terminal of L K A M D P T P P L (Leu Lys Ala Met Asp Pro Thr Pro Pro Leu—SEQ ID NO 1 herein). Another version designated LT-15 consisting of 15 amino acids and a sequence from the N-terminal of L K A M D P T P P L W I K T E (Leu Lys Ala Met Asp Pro Thr Pro Pro Leu Trp Ile Lys Thr Glu—SEQ ID NO 2 herein); and another version designated LT-5 consisting of 5 amino acids and a sequence from the N-terminal of L K A M D (Leu Lys Ala Met Asp—SEQ ID NO 3 herein) were also made. All three versions; LT-15, LT-10 and LT-5 have similar biological activity and are useful in this invention as are the peptides of intermediate length. For convenience, the invention is largely described hereinafter with reference to LT-10, although the invention should not be construed as being so limited.

### BRIEF DESCRIPTION OF THE DRAWINGS

FIG. 1 graphically illustrates experimental results obtained from certain of the examples.

### DETAILED DESCRIPTION OF THE INVENTION

In one embodiment of the invention, there is provided a method for assaying human endogenous proteins from saliva. A saliva sample is obtained and an ELISA assay performed on the sample employing an anti-serum which is specific for the protein of interest.

Useful information is obtained by analyzing for at least one of IgE, NGF, Insulin, Myoglobin and ADA. The ELISA is performed with anti-IgE, anti-NGF, anti-Insulin, anti-Myoglobin, and anti-ADA, as applicable.

Elevated levels of serum proteins selected from the group consisting of free IgE, NGF, Insulin, Myoglobin and ADA can be reduced by administering to said human exhibiting such. level an effective amount of a peptide containing at least the first four amino acids from the N-terminal of the sequence Leu Lys Ala Met Asp Pro Thr Pro Pro Leu Trp Ile Lys Thr Glu (SEQ ID NO: 2). Preferably, the-peptide contains the sequence of at the least first four amino acids beginning at its N-terminal and has no more than 20 amino acids total, and more preferably has in the range of from five, to fifteen amino acids total. Most preferably, the peptide has from eight to 12 amino acids total and is selected from the group of peptides Leu Lys Ala Met Asp Pro Thr Pro Pro Leu Trp Ile (SEQ ID NO.4), Leu Lys Ala Met Asp Pro Thr Pro Pro Leu Trp (SEQ ID NO .5), Leu Lys Ala Met Asp Pro Thr Pro Pro Leu (SEQ ID NO 1), Leu Lys Ala Met Asp Pro Thr Pro Pro (SEQ ID NO 6), and Leu Lys Ala Met Asp Pro Thr Pro (SEQ ID NO 7).

By using peptides as described above, the peptide can be and preferably is orally administered and serum free IgE level is reduced.

Generally speaking, in the range of from about 0.02 to about 200 milligrams of the peptide is orally administered on a daily basis, usually in the range of from about 0.2 to about 20 milligrams on a daily basis. Oral administration of an amount of the 10 amino acid peptide within the range of 0.2 to 5 milligrams daily has been demonstrated to markedly influence blood protein levels, and an amount in the range of 0.5 to about 2 milligrams daily has been tested with good results. Usually, the peptide is administered to humans having an elevated serum free IgE level, as compared to norms. Often, a patient having an elevated free IgE level will also have an elevated NGF, Insulin, Myoglobin and/or ADA serum level.

The peptide is believed effective to treat conditions selected from the group consisting of Asthma, Diabetes, Depression and Autoimmune Disease. Typical autoimmune diseases are selected from the group consisting of erythematosus (SLE), Rheumatoid arthritis, Sjogren's syndrome, Reiter's syndrome, Graves' disease, Addison's disease, and Hodgkin's disease.

The proposed treatment with LT-10 to lower the concentration of free IgE has several advantages over-the contemplated use of monoclonal antibodies against IgE (Mono anti-IgE). LT-10 is a synthetic peptide made of 10 amino acids, which can be made in abundance and very chiefly. Mono anti-IgE is a big protein molecule and the cost can be $ 3,000 to 5,000 per mg. LT-10 can be given orally under the tongue. Mono anti-IgE must be given by injection only. Being a large molecule, it will not be absorbed by oral administration. Both LT-10 and Mono anti-IgE neutralize the circulating IgE and lower the free IgE level. Excess LT-10 in the system will not do any harm However, excess of Mono anti-IgE unused will start making antibodies. These anti idiotypic antibodies or anti-anti Mono IgE, which is a copy of IgE, will interfere with treatment, We propose LT-10 treatment should be continuous in order to maintain free IgE level to normal state. Because, IgE level is known to rise under environmental, emotional stress and exercise etc., mono anti-IgE treatment can not be given continuously due route of delivery and expense etc.

Experimental

Following experiments were performed.

Experiment 1: The pool of several human salivas was split into two parts. To one part equal volume of PBS was added and to the second part equal volume-containing 1 mg/ml of LT-10 was added. The mixtures were incubated at 37 ° C. for one hour. IgE levels were assayed in both mixtures by usual ELISA test using anti-IgE. It was revealed that free IgE level was much reduced in the mixture of saliva and LT-10, in comparison to the mixture of saliva and PBS. This shows the binding of LT-10 to free IgE in saliva, the bound IgE is not detected by anti-IgE by ELISA test.

Experiment 2: 1 placed one ml of water in my mouth and kept it for 15 minutes, after which the mixture with saliva and water was collected Likewise I placed one ml of LT-10 containing 1 mg/ml and the mixture of saliva and LT-10 was collected. IgE levels were assayed in both mixtures by usual ELISA test. It was revealed that IgE level was much reduced in the mixture of saliva and LT-10, in comparison to the mixture of saliva and water. This shows that the binding of LT-10 to IgE in saliva in mouth.

Experiment 3: A serum pool from allergy patients was divided into a first tube and a second tube. PBS was added to the first tube, and LT-10 solution was added to the second, the serum concentration in both tubes being the same. After incubation, both tubes were analyzed for IgE concentration by using anti-IgE. The concentration of free IgE in the tube treated with LT-10 was lower than in the untreated tube.

Experiment 4: LTNF has been applied topically to a human to treat a bee sting which had become swollen and stiff. The itching stopped immediately and the inflammation and stiffness were reduced soon thereafter.

Experiment 5: LTNF was applied topically to a human to treat a skin allergy and provided immediate relief from the itching caused by the allergy.

Experiment 6: LTNF was applied topically to the hands of a human suffering from arthritis. The pain was relieved shortly after the application of the LTNF.

Experiment 7: A person suffering from asthma took LTNF by nasal insufflation and experience relief with sinus clearance in a short time. Another person with a long term history of being unable to breathe through their nose took LTNF by nasal insufflation and experienced relief in three days.

The prior art has advocated anti-IgE treatment only for allergic rhinitis and asthma. After discovering the high levels of IgE implicated for other than asthma disorders, we advocate LT-10 treatment for the disorders where IgE levels are high, those are: (1) Type II diabetes (2) Depression (3) various types of Autoimmune disorders and (4) Asthma.

Currently, diabetes, depression and autoimmune diseases are treated with various drugs. For example, diabetes treated by insulin injections, and depression with anti depression drugs like Prozac. Autoimmune disorders are treated with immuno-suppressive drugs. We obtained saliva from the people who are undergoing treatment for their respective disorders for years. Our results emphasize that in spite of the conventional treatment, IgE levels remained very high causing disruption in homeostasis of other proteins, which were also high, despite the patients having taken medications for a long time. The elevated levels of NGF, myoglobin, insulin, and ADA, are measured in saliva of the people having high concentration of IgE indicating damage of various organ

LT-10 treatment lowers the free IgE level and the levels of other measured proteins. We believe that LT-10 treatment is ideal for these diseases and LT-10 has no observable side effects.

Human Saliva: Saliva from individual was collected in a centrifuge tube. Collected saliva was centrifuged and the supernatant was separated. Protein concentration of the saliva was measured by spectrophotometer. The protein content for saliva was adjusted to 200 μg/ml and stored frozen from which it was diluted in carbonate-bicarbonate buffer pH 9.4 to give the concentration. 10 μg/ml for ELISA tests.

Following antisera were used to assay IgE, NGF, Insulin, Myoglobin and ADA. Anti-IgE, and anti-NGF were made in house, by immunizing rabbit. Anti-Myoglobin made in rabbits was purchased from OEM concepts; Anti-insulin made in pig was purchased from Sigma-Aldrich Co. Anti-ADA is not available commercially was made in house by immunizing BALB/c mice.

Enzyme-Linked Immunosorbent Assay (ELISA) for human saliva:

ELISA tests were performed in 96 well micro-plate. The wells of the plate were coated with saliva at 10 μg/ml concentration in carbonate-bicarbonate buffer pH 9.4, each well receiving 00 μl. After overnight incubation at room temperature the plate was washed three times with 0.05 phosphate buffered saline (PBS). Anti-IgE diluted in 3% gelatin from 1:100 to 1:2187 was added to three wells for each dilution. Similar procedure was followed for assaying NGF, myoglobin insulin and ADA by using respective anti-sera; such as anti-NGF, anti-myoglobin; anti-insulin and anti-AD. Antigen-antibody reaction was carried at.37° C. for 1.5 hours. After which the plate-was washed and was reacted with horseradish peroxidase conjugated with IgG. Rabbit horseradish peroxidase was reacted for rabbit anti-IgE and anti-NGF; pig peroxidase for pig anti-insulin and mouse peroxidase for-mouse anti-ADA.

Assays of endogenously present proteins, IgE, NGF, Myoglobin, Insulin and ADA in human saliva are compared with the normal controls. The results are presented in tables 1 and 2. The ELISA titers for IgE, NGF, myoglobin, Insulin and ADA were divided by a normal ELISA titer, to give the normalized reading.

TABLE 1
High Level of IgE corresponds to high levels
of NGF and Myoglobin in human saliva.

| | | | IgE/ | | NGF/ | | Myo/ |
|---|---|---|---|---|---|---|---|
| Specimen | Status | IgE | Norm | NGF | Norm | Myo | Norm |
| Pool of 6 | Normal | 12150 | 1.00 | 1200 | 1.00 | 1800 | 1.00 |
| Pool of 2 | Marginal | 32400 | 2.67 | 1800 | 1.50 | 2700 | 1.50 |
| Pool of 2 | Diabetes | 145800 | 12.00 | 5400 | 4.50 | 3600 | 2.00 |
| J C | Diabetes | 145800 | 12.00 | 24300 | 20.25 | 10800 | 6.00 |
| T F | Asthma | 145800 | 12.00 | 5400 | 4.50 | 5400 | 3.00 |
| W K | Depres- | 218700 | 18.00 | 24300 | 20.25 | 16200 | 9.00 |
| | sion | | | | | | |
| W C | Normal | 16200 | 1.33 | 2700 | 2.25 | 1800 | 1.00 |
| R C | Auto-imm | 72900 | 6.00 | 5400 | 4.50 | 3600 | 2.00 |
| B S | Auto-imm | 218700 | 18.00 | 8100 | 6.75 | 10800 | 6.00 |
| R G | Auto-imm | 48600 | 4.00 | 1800 | 1.50 | 1800 | 1.00 |
| A A | Auto-imm | 72900 | 6.00 | 2700 | 2.25 | 3600 | 2.00 |
| S G | Auto-imm | 72900 | 6.00 | 8100 | 6.75 | 5400 | 3.00 |
| R C | Auto-imm | 437400 | 36.00 | 24300 | 20.25 | 32400 | 18.00 |
| J C | Auto-imm | 437400 | 36.00 | 24300 | 20.25 | 32400 | 18.00 |
| V A | Auto-imm | 48600 | 4.00 | 2700 | 2.25 | 1800 | 1.00 |
| G A | Auto-imm | 437400 | 36.00 | 16200 | 13.50 | 32400 | 18.00 |
| N G | Auto-imm | 48600 | 4.00 | 2700 | 2.25 | 5400 | 3.00 |
| Normal | | 12150 | | 1200 | | 1800 | |

Table 1

Results of Table 1 show that:

(1) IgE levels are higher than normal in saliva from diabetes, asthma, depression and various types of autoimmune disorders. IgE level varied from 2.67 times as in the marginal normal people to 36 times as in autoimmune disorder patients in comparison to normal counterpart.

(2) Patients showing high levels of IgE showed high levels of NGF. NGF levels varied from 4.5 times in diabetes to 20.25 times in depression and autoimmune disorders.

(3) Patients showing high levels of IgE showed high levels of myoglobin. Myoglobin levels varied from 3.0 times in asthma patient to 18.0 times in autoimmune disorders.

TABLE 2
High Level of IgE corresponds to high levels of
Insulin and ADA in human saliva.

| | | | IgE/ | | Ins/ | | ADA/ |
|---|---|---|---|---|---|---|---|
| Specimen | Status | IgE | Norm | Insulin | Norm | ADA | Norm |
| Pool of 6 | Normal | 12150 | 1.00 | 450 | 1.00 | 600 | 1.00 |
| Pool of 2 | Marginal | 32400 | 2.67 | 600 | 1.33 | 900 | 1.50 |
| Pool of 2 | Diabetes | 145800 | 12.00 | 1800 | 4.00 | 1800 | 3.00 |
| J C | Diabetes | 145800 | 12.00 | 1800 | 4.00 | 1800 | 3.00 |
| T F | Asthma | 145800 | 12.00 | 2700 | 6.00 | 8100 | 13.5 |
| W K | Depres- | 218700 | 18.00 | 1800 | 4.00 | 2700 | 4.50 |
| | sion | | | | | | |
| W C | Normal | 16200 | 1.33 | 300 | 0.67 | 600 | 1.00 |
| R C | Auto-imm | 72900 | 6.00 | 450 | 1.00 | 2700 | 4.50 |
| B S | Auto-imm | 218700 | 18.00 | 2700 | 6.00 | 2700 | 4.50 |
| R G | Auto-imm | 48600 | 4.00 | 450 | 1.00 | 450 | 0.75 |
| A A | Auto-imm | 72900 | 6.00 | 450 | 1.00 | 450 | 0.75 |
| S G | Auto-imm | 72900 | 6.00 | 2700 | 6.00 | 450 | 0.75 |
| R C | Auto-imm | 437400 | 36.00 | 2700 | 6.00 | 2700 | 4.50 |
| J C | Auto-imm | 437400 | 36.00 | 2700 | 6.00 | 1800 | 3.00 |
| V A | Auto-imm | 48600 | 4.00 | 900 | 2.00 | 900 | 1.50 |
| G A | Auto-imm | 437400 | 36.00 | 1800 | 4.00 | 1800 | 3.00 |
| N G | Auto-imm | 48600 | 4.00 | 900 | 2.00 | 900 | 1.50 |
| Normal | | 12150 | | 450 | | 600 | |

Results of table 2 show

(1)IgE levels are higher than normal in saliva from diabetes, asthma, depression and various types of autoimmune disorders. IgE level varied from 2.67 times as in the marginal normal people to 36 times as in autoimmune disorders patients in comparison to normal counterpart.

(2) Patients showing high levels of IgE showed high levels of Insulin. Insulin levels varied from 4.0 times in diabetes patient to 6.0 times in autoimmune disorders.

(3) Patients showing high levels of IgE showed high levels of ADA especially in asthma patient, 13.5 times greater than normal. Some autoimmune patients showed lower level of ADA in comparison to normal people. Thus ADA level varied from 0.7 to 6 times.

Collectively, the results of Tables 1 and 2 clearly show that the elevated level of IgE is the culprit—causing numerous types of disorders. The elevated level caused increased levels for other proteins such as NGF, Myoglobin, insulin and in case of asthma ADA.

Personal Example from the Inventor Binie Lipps:

On my annual medical check, I was diagnosed to diabetes based on the high level of glucose in blood, the only available test for diagnosis. I did not have discomfort or symptoms. I took Glucotrol treatment for two months as was prescribed by the doctor. After two months of Glucotrol treatment and sugar-free diet, the blood glucose level came down but remained high. I often used to get allergic reactions. Therefore, I realized that high glucose in blood may be related to allergic reaction. In the meantime, I discovered that IgE can be assayed from salvia. Before that, an assay of IgE was possible only from an invasive procedure to obtain a serum specimen. I also discovered that the endogenously present other proteins, NGF, Myoglobin, Insulin and ADA can be assayed from saliva by ELISA test.

After the discovery that IgE could be assayed from saliva, the following experiments were performed. Fasting saliva collected and glucose level measured for seven days for each experiment. Sugar free diet was observed during all experiments. In addition to IgE, NGF, Myoglobin, Insulin and ADA were assayed in saliva. After completion of an experiment two day waiting period was allowed before starting the next experiment.

Experiment #1: No treatment.

Experiment #2: Glucotrol treatment, 10 mg in the morning and 5 mg in the evening.

Experiment #3: LT-10 treatment 2 mg/day, 1 mg in the morning and 1 mg in the evening

Experiment #4. Combination of LT-10, 2 mg/day and 15 mg/day Glucotrol.

results of these experiments are shown in tables 3 to 7.

TABLE 3
Blood Glucose level in mg:

Treatment

| | None | Gluco | LT-10 | Combination |
|---|---|---|---|---|
| Day | Expt#1 | Expt#2 | Expt#3 | Expt#4 |
| 1 | 305 | 183 | 132 | 137 |
| 2 | 244 | 183 | 124 | 145 |
| 3 | 144 | 209 | 116 | 140 |
| 4 | 186 | 199 | 123 | 142 |
| 5 | 203 | 218 | 151 | 150 |
| 6 | 191 | 208 | 183 | 158 |
| 7 | 116 | 214 | 155 | 150 |

The results show that the glucose level remained variable in all four experiments. In expt #1 sugar level fluctuated from 116 to 301. In expt #2 glucose level fluctuated from 183 to 214, with Glucotrol treatment, did not make appreciable difference for glucose. However, in experiment #3 and in expt #4 the glucose levels remained lower in comparison to expt #1 and #2. Fluctuation in expt #3 was 116 to 183 and in expt #4 137 to 158. Glucotrol treatment may be lower glucose level as in exp#2. However, it is not Glucotrol but LT-10 lowered the glucose level as in expt #3 and #4.

TABLE 4
IgE levels in saliva:

Treatment

| | None | Gluco | LT-10 | Combi |
|---|---|---|---|---|
| Day | Expt#1 | Expt#2 | Expt#3 | Expt#4 |
| 1 | 145800 | 145800 | 145800 | 145800 |
| 2 | 148600 | 148600 | 72900 | 145800 |
| 3 | 148600 | 145800 | 72900 | 145800 |
| 4 | 148600 | 148600 | 72900 | 72900 |
| 5 | 145800 | 148600 | 72900 | 72900 |
| 6 | 145800 | 145800 | 72900 | 48600 |
| 7 | 145800 | 145800 | 48600 | 24300 |

Normal 16200

IgE levels remained high in expt # 1 and 2 with no treatment or Glucotrol treatment. LT-10 treatment along for seven days as in expt #3 or in combination with Glucotrol as in expt #4 lowered the IgE levels almost reaching to normal. Results clearly indicate that Glucotrol does not contribute in lowering IgE levels. It is the LT-10 treatment which causes the lowering of IgE.

TABLE 5
NGF levels in saliva:

Treatment

| | None | Gluco | LT-10 | Combi |
|---|---|---|---|---|
| Day | Expt#1 | Expt#2 | Expt#3 | Expt#4 |
| 1 | 2700 | 2700 | 2700 | 2700 |
| 2 | 2700 | 8100 | 2700 | 3600 |
| 3 | 2700 | 5400 | 2700 | 3600 |
| 4 | 2700 | 5400 | 1800 | 3600 |
| 5 | 2700 | 2700 | 1800 | 2700 |
| 6 | 5400 | 2700 | 1800 | 2700 |
| 7 | 5400 | 5400 | 1800 | 2700 |

Normal 1200

NGF levels remained high in expt #1 and 2 with no treatment or Glucotrol treatment. LT-10 treatment alone for seven days as in expt # 3 lowered the NGF, levels almost to normal. It seems that as in expt #2 with Glucotrol alone and in expt #4 the combination of LT-10 and Glucotrol caused elevation in NGF. Results clearly indicate that Glucotrol does not contribute in lowering NGF levels. On the contrary, Glucotrol perhaps increases NGF levels. LT-10 treatment causes the lowering of NGF to bring normal homeostasis.

TABLE 6
Insulin levels in saliva:

Treatment

| | None | Gluco | LT-10 | Combi |
|---|---|---|---|---|
| Day | Expt#1 | Expt#2 | Expt#3 | Expt#4 |
| 1 | 2700 | 1200 | 2700 | 2700 |
| 2 | 2700 | 800 | 1800 | 1800 |
| 3 | 1800 | 800 | 1800 | 2700 |
| 4 | 1200 | 900 | 1800 | 2700 |
| 5 | 1800 | 750 | 1800 | 1800 |
| 6 | 1800 | 800 | 1800 | 900 |
| 7 | 2700 | 900 | 900 | 900 |

Normal 600

Insulin levels remained high in expts #1 and 2 with no treatment or Glucotrol treatment. LT-10 treatment alone for seven days as in expt #3 or in combination with Glucotrol as in expt #4 lowered the Insulin levels to almost normal. Results indicate that perhaps Glucotrol treatment contributes m lowering Insulin levels as expts #2 and 4.

TABLE 7
Myoglobin levels in saliva:

Treatment

| | None | Gluco | LT-10 | Combi |
|---|---|---|---|---|
| Day | Expt#1 | Expt#2 | Expt#3 | Expt#4 |
| 1 | 1800 | 1800 | 1800 | 2700 |
| 2 | 1800 | 3600 | 1800 | 3600 |
| 3 | 3600 | 3600 | 2700 | 3600 |
| 4 | 3600 | 2700 | 1800 | 3600 |
| 5 | 1800 | 2700 | 1800 | 2700 |
| 6 | 2700 | 2700 | 1800 | 2700 |
| 7 | 1800 | 3500 | 1800 | 2700 |

Normal 1800

Myoglobin levels remained high in expts #1 and 2 with no treatment or Glucotrol treatment. LT-10 treatment alone for seven days as in expt #3 lowered the myoglobin levels to almost normal. Results indicate that perhaps Glucotrol treatment contributes in increasing myoglobin levels as seen in expts #2 and 4. This side effect of Glucotrol treatment may be implicated to heart trouble.

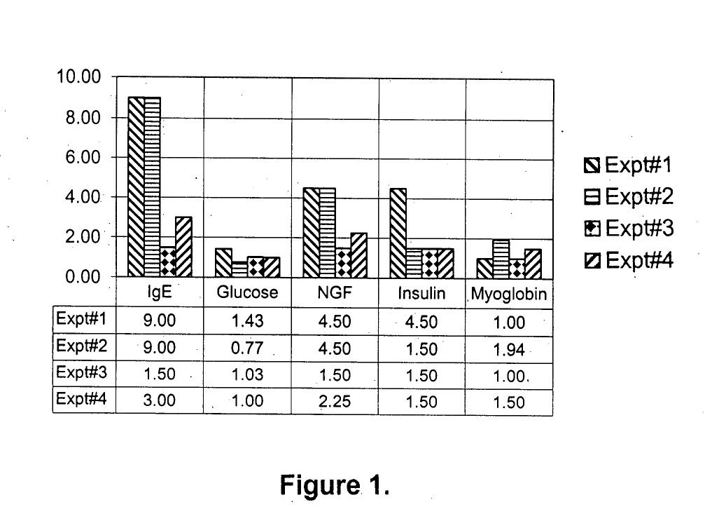

In FIG. 1, Expt #1 is no treatment, Expt #2 is Glucotrol treatment, Expt #3 is LT-10 treatment and Expt #4′is Glucotrol+LT-10. The levels of IgE, Glucose, NGF, Insulin and myoglobin are expressed as times the normal level of the respective protein.

The results of the four experiments at the completion point which is the end of seven days are graphically illustrated in FIG. 1:

1. IgE level remained high in expts #1 and #2. Lowered by LT-10 treatment as in expt #3 and with combination treatment.

2. Glucose level responded variably in all experiments. Decrease in glucose occurred in experiments 2, 3 and 4.

3. NGF level remained high at the end of expts #1 and #2. LT-10 alone or in combination with Glucotrol as in expts #3 and #4 lowered the NGF level. Glucotrol NGF which increases inflammation.

4. Insulin level decreased in all three expts #2, 3, 4.

5. Glucotrol treatment alone or in combination with LT-10 increased the level of myoglobin. This may be a side effect of Glucotrol giving heart problems.

What is claimed is:

1. A method for reducing serum levels of free IgE in a human having an elevated level of free IgE, said method comprising determining that said human has an elevated level of free IgE in their serum, and administering to said human an effective amount of a peptide selected from the group consisting of
SEQ. ID. NO.: 4,
SEQ. ID. NO.: 5,
SEQ. ID. NO: 1,
SEQ. ID. NO.: 6, and
SEQ. ID. NO.: 7,
to reduce the level of free IgE in the serum of said human.

2. A method as in claim 1 wherein the peptide consists of SEQ. ID. NO.: 1.

3. A method as in claim 2 wherein the peptide is orally administered.

4. A method as in claim 2 wherein the range of from about 0.02 to about 200 milligrams of the peptide are orally administered on a daily basis.

5. A method as in claim 2 wherein in the range of from about 0.2 to about 20 milligrams of the peptide is ovally administered on a daily basis.

6. A method as in claim 2 wherein said human has an elevated serum level of unbound IgE prior to the step of administering the peptide.

7. A method as in claim 6 wherein the peptide binds a portion of the free IgE and reduces the serum level of free IgE.

8. A method as in claim 7 wherein said human further has an elevated serum level of NGF, Insulin, Myoglobin and/or ADA prior to the step of administering the peptide and said elevated serum level of NGF, Insulin, Myoglobin and/or ADA is reduced following the step of administering the peptide.

9. A method as in claim 6 further comprising assaying a saliva IgE level in said human.

10. A method as in claim 8 wherein said human further has a condition selected from the group consisting of
Asthma, Diabetes, and allergy.

11. A method of inhibiting an immunological reaction between human IgE and human anti-IgE comprising contacting the human IgE with a a peptide selected from the group consisting of
SEQ. ID. NO.: 4,
SEQ. ID. NO.: 5,
SEQ. ID. NO.: 1,
SEQ. ID. NO.: 6, and
SEQ. ID. NO.: 7
to form a complex and bringing the complex together with the human anti-IgE.

12. A method as in claim 11 wherein the human IgE is contained in serum.

13. A method as in claim 11 wherein the human IgE is contained in saliva.

14. A method as in claim 11 wherein the peptide consists of SEQ. ID. NO.: 1.

15. A method for binding human IgE in a patient, said method comprising selecting a patient having an elevated level of IgE and a condition selected from the group consisting of asthma, type 2 diabetes, and allergic reaction, and orally administering SEQ. ID. NO.: 1 said patient, whereby a portion of the human IgE becomes bound to SEQ. ID. NO.: 1 and is rendered undetectable by ELISA employing anti-human-IgE.


SEQUENCE LISTING (7 sequences)

SEQ ID NO: 1 - protein, 10 aa, Artificial
  Synthesized.  Active fragment of isolate from opossum serum.  See US 5,576,297
LKAMDPTPPL

SEQ ID NO: 2 - protein, 15 aa, Artificial
  Synthesized.  Active fragment of isolate from opossum serum.  See US 5,576,297.
LKAMDPTPPLWIKTE

SEQ ID NO: 3 - protein, 5 aa, Artificial
  SYNTHESIZED.  ACTIVE FRAGMENT OF ISOLATE FROM OPOSSUM SERUM.  SEE US 5,576,297.
LKAMD

SEQ ID NO: 4 - protein, 12 aa, Artificial
  SYNTHETIC.  CORRESPONDS TO FRAGMENT 1-12 OF 2 ABOVE.
LKAMDPTPPLWI

SEQ ID NO: 5 - protein, 11 aa, Artificial
  SYNTHETIC.  CORRESPONDS TO FRAGMENT 1-11 OF 2 ABOVE.
LKAMDPTPPLW

SEQ ID NO: 6 - protein, 9 aa, Artificial
  SYNTHETIC.  CORRESPONDS TO FRAGMENT 1-9 OF 2 ABOVE.
LKAMDPTPP

SEQ ID NO: 7 - protein, 8 aa, Artificial
  SYNTHETIC.  CORRESPONDS TO FRAGMENT 1-8 OF 2 ABOVE.
LKAMDPTP